(19) United States
(12) Patent Application Publication
(10) Pub. No.: US 2025/0099223 A1
(43) Pub. Date: Mar. 27, 2025

(54) ACCESS DEVICE, SYSTEM, AND METHOD FOR END-SIDE ANASTOMOSIS TO PERIPHERAL VASCULALTURE

(71) Applicants: Connex Biomedical, Inc., Aurora, CO (US); The Board of Regents of University of Colorado, a body corporate, Denver, CO (US)

(72) Inventors: Max Bannister Mitchell, Castle Pines, CO (US); Jeremy H. Morgan, Missoula, MT (US); Jeffrey N. Steinmetz, Arvada, CO (US)

(73) Assignees: Connex Biomedical, Inc., Aurora, CO (US); The Board of Regents of University of Colorado, a body corporate, Denver, CO (US)

(21) Appl. No.: 18/890,048

(22) Filed: Sep. 19, 2024

Related U.S. Application Data
(63) Continuation of application No. 18/188,390, filed on Mar. 22, 2023.
(63) Continuation-in-part of application No. 18/188,393, filed on Mar. 22, 2023.
(60) Provisional application No. 63/583,751, filed on Sep. 19, 2023.
(60) Provisional application No. 63/583,852, filed on Sep. 19, 2023.

(51) Int. Cl. A61F 2/06; A61B 17/11

(52) U.S. Cl. CPC A61F 2/064; A61B 17/11; A61B 2017/1107; A61B 2017/1135; A61F 2220/0016

(57) ABSTRACT

A vascular access device and assembly characterized by a tubular access graft having at least one angled distal flange that forms a non-circular distal opening to the tubular access graft, with the angled distal flange having a reinforcement structure that expands the flange and abuts a luminal wall surface of a blood vessel. Optionally, an axial compression ring or cuff may be provided that concentrically engages the tubular access graft and is translatable into a position that abuts an abluminal wall surface of the blood vessel. Also optionally, the device and assembly may include an annular ring with a plurality of axially projecting tines arrayed about a circumference of the annular ring, the annular ring being adapted to evert upon engagement with vascular tissue and creation of an access open by either an arteriotomy or a venotomy. The plurality of tines are configured to engage vascular tissue and then rotate to a different axial orientation to evert the periphery of the access site. The plurality of tines may be used to couple a graft in an end-to-side anastomosis or be used without a graft to reinforce the access site and then allow for closure of the access site. The vascular access device and assembly is particularly useful in providing axillary artery access for introducing a medical device, such as a catheter based axial flow ventricular assist device, into and through the axillary artery and into the heart.

### CROSS-REFERENCE TO RELATED APPLICATIONS

This application claims priority to U.S. Provisional Patent Application Ser. Nos. 63/583,751 and 63/583,852, both filed Sep. 19, 2023, and is a continuation-in-part of co-pending U.S. patent application Ser. Nos. 18/188,390 and 18/188,393, both filed Mar. 22, 2023.

### BACKGROUND OF THE INVENTION

The present disclosure pertains to access devices and systems for placement of medical devices into and through the peripheral vasculature. More particularly, the present disclosure pertains to access devices and systems for introduction, placement, and withdrawal of circulatory assist devices into and through the peripheral vasculature, arterial cannulation for cardiopulmonary bypass, extra corporeal membrane oxygenation (ECMO), and/or bypass grafting. Still more particularly, the present disclosure pertains to access devices and systems for introduction and placement of circulatory assist devices, such as a catheter-based axial flow ventricular assist device (AF-VAD) into and through the axillary, innominate, iliac, or common femoral arteries and into a chamber of the heart, followed by withdrawal of the AF-VAD from the heart and axillary artery and closure of the arterial access site.

Access to the peripheral vasculature, such as, for example, the axillary artery, requires either percutaneous or surgical access to the blood vessel. Where percutaneous access is performed, access may be achieved such as by the Seldinger technique with guidewire guidance. Where surgical access is performed, a small incision is made to access the blood vessel, then an arteriotomy is performed to access the vascular lumen of the blood vessel.

Once access is obtained, an end-to-side anastomosis is performed to join the disclosed access device and system to the blood vessel.

The access devices and systems according to the present disclosure have multiple variants. In accordance with a first variant, there is disclosed a tubular access graft that is configured to couple to an anastomosis site and is secured at the anastomosis site at least one reinforced flange at a distal end of the access graft wherein reinforcement in the flange expands the flange and abuts a luminal wall surface of a blood vessel and/or by having an anastomosis connector. In accordance with a second variant, there is provided a sealing access port that couples to an anastomosis site, wherein the port has a central opening with a seal that allows a medical device, such as an AF-VAD to pass into and through the central opening, reside within the opening in a hemostatic manner, and allows for withdrawal of the medical device through the central opening and sealing the anastomosis site post-procedure.

The first variant of the access device and system of the present disclosure has several sub-variants. A first sub-variant includes a reinforced flanged graft in which there is at least one distal flange that is angularly displaced from the longitudinal axis of the tubular graft, such as having about a 30-degree to about a 40-degree angle, and a support ring. The flange is reinforced with any of a wide variety of reinforcements, including, for example, a latticed stent-like structure that is either coupled to or encapsulated within the flange, at least one wire that is either coupled to or encapsulated within the flange, a wire hoop coupled to at least a portion of the outer perimeter of the flange with the wire hoop being either an elastic or shape memory material that is configured to fold upon itself and then deploy into either a circumferential reinforcement of the flange or as figure-8 or lemniscate configuration in which the wire overlaps itself intermediate opposing open loop the figure-8 or lemniscate structure, when delivered into the arteriotomy to expand the flange and seat the flange within the blood vessel.

The at least one distal flange is adapted to nest, at least partially, against the luminal wall of the blood vessel, for example by assuming a generally saddle-shape under the influence of the reinforcing member and upon being released within the blood vessel. Once the distal flange is abutting the luminal surface of the blood vessel, an optional axial compression ring or cuff may be provided that is placed over the access graft and placed against the abluminal wall of the blood vessel. The axial compression ring or cuff seats exerts an axial pressure against the abluminal wall of the blood vessel and axially tensions the distal flange against the luminal wall of the blood vessel, thereby sealing the arteriotomy between the distal flange and the axial compression ring or cuff.

The reinforcement member of the distal flange may be coupled to a peripheral surface of the distal flange, may be coupled to the radial surface(s) of the distal flange, or may be embedded or encapsulated in the material of the distal flange. The reinforcement member May be a pliable polymeric material that has a greater durometer and/or a lower modulus of elasticity than the material of the distal flange or may be a shape memory material capable of being shape set to a desired configuration.

The at least one distal flange may consist of a unitary circumferential flange, a unitary partially circumferential flange. Alternatively, the at least one distal flange may consist of two diametrically extending and opposing flanges, with a first flange extending in a proximal orientation and a second flange extending in a distal orientation relative to a longitudinal axis of the blood vessel. The first and second flanges may be symmetrical or asymmetrical with each other. Where two diametrically extending and opposing flanges are asymmetrical, the two flanges will have different configurations with the first flange having a different configuration than the second flange. Where the first and second flanges are asymmetrical, the first flange extending proximally in the blood vessel will preferably have a larger profile to cover a larger luminal surface area of the blood vessel, such as greater than a 180-degree arc about the circumference of the blood vessel, as it projects proximally relative to blood flow through the blood vessel and serves as a guidance path for a medical device introduced into the blood vessel through the access device. Further, where the first and second flanges are asymmetrical, the second flange will have a smaller profile that the first flange and project distally relative to blood flow through the blood vessel and cover a smaller luminal surface area of the blood vessel, such as less than a 180-degree arc about the circumference of the blood vessel, than the first flange. As an example of an asymmetric flange, the first flange may extend proximally along a length of the blood vessel and extend about a substantial or entire circumferential aspect of the blood vessel with the reinforcing member and the first flange forming a tubular section of the first flange. Where the distal flange is angularly displaced from the longitudinal axis of the access graft, it is preferable that the angular displacement be oriented such that the access graft enters the blood vessel with an orientation dictated by the device being passed through the access graft. For example, where an AF-VAD is introduced through the access graft and into the axillary artery, the angular orientation of the access graft will be such that it facilitates passage of the AF-VAD into the axillary artery proximally relative to the blood flow to direct passage of the AF-VAD upstream in the blood flow toward the aorta.

The axial compression ring or cuff may have a wide variety of configurations, including, for example a shape memory wire that is formed into a semi-circular, semi-ovular, or semi-elliptical shape and a shape memory tube that receives opposing ends of the shape memory wire within the lumen of the shape memory tube to allow the diametric expansion and contraction of the shape memory wire. Alternatively, the axial compression ring or cuff may be an elastic collar, band, or annular ring that is concentrically engageable about the access graft. Further, the axial compression ring or cuff may be a rigid or semi-rigid tubular or annular member having a distal flange that is configured to abut the abluminal surface of the blood vessel. The axial compression ring or cuff may further have a structure, such as threads, detents, projections, or other structures that operably interface with mating structures on the access graft. For example, the access graft may have helical projections on an exterior surface thereof that engage the structures on the axial compression ring or cuff to allow the axial compression ring or cuff to rotate about the access graft until abutting the abluminal surface of the blood vessel. As a further example, the access graft may have circumferential rings or detents that engage with mating structures on the axial compression ring or cuff and allow for translation of the axial compression ring or cuff along a length of the access graft until the axial compression ring or cuff is abutting the abluminal surface of the blood vessel.

It will be appreciated that with an asymmetric flange having proximal and distal flanges the first flange forms a toe portion and the second flange forms a heel portion of the distal flange. By configuring the first flange as a toe portion extending proximally relative to the blood flow, a medical device, such as an AF-VAD, may be introduced through the access graft, pass into the blood vessel, e.g., the axillary artery, and be delivered upstream in the blood flow to the heart where it will perform its function as a circulatory assist device for a period of time. Once the circulatory assist device is no longer required, the AF-VAD is removed in a retrograde manner through the blood vessels and out the access graft. After removal from the access graft, the access graft is either closed or removed surgically with closure of the arteriotomy.

### SUMMARY OF THE INVENTION

It is an object of the present disclosure to provide an access device and assembly configured to allow introduction of a medical device into the peripheral vasculature, to allow the medical device to reside for a period of time within the vasculature, allow withdrawal of the medical device from the vasculature, and allow for closure of or removal of the access device and assembly.

It is another object of the present disclosure to provide an access device and assembly in which there is provided an access graft having a reinforced distal flange, wherein the reinforced distal flange is placed within a blood vessel and abuts against a luminal wall surface of the blood vessel to secure the access graft with the blood vessel and create an access pathway through the access graft and into the blood vessel for introduction of a medical device into the blood vessel.

It is still another object of the present disclosure to provide the reinforced distal flange such that it has an angular orientation relative to the access graft and forms a non-circular transverse cross-sectional opening at a distal end of the access graft and an angular approach to the access graft relative to the longitudinal axis of the blood vessel.

It is yet another object of the present disclosure to provide an access graft and assembly in which the reinforced flange has an associated structural support that diametrically expands the reinforced flange within the lumen of the blood vessel and seats the reinforced flange in abutting relationship with a luminal wall surface of the blood vessel.

It is a further object of the present disclosure to provide an access graft and assembly in which the reinforced flange further has two diametrically opposing and projecting reinforced flanges, with a first flange projecting proximally and a second flange projecting distally relative to the blood flow through the blood vessel.

It is another further object of the present disclosure to provide the first flange with a structural support that reinforces the first flange and supports the first flange in an abutting relationship with the luminal wall surface of the blood vessel about a circumferential arc greater than 180-degrees about the circumference of the luminal wall surface of the blood vessel.

It is still another further object of the present disclosure to provide the second flange with a structural support that reinforces the second flange and supports the second flange in an abutting relationship with the luminal wall surface of the blood vessel about a circumferential arc less than 180-degrees about the circumference of the luminal wall surface of the blood vessel.

It is yet another object of the present disclosure to provide the distal flange with a structural support that is joined to proximal and distal portions of the distal flange but not the lateral portions of the distal flange.

It is yet another further object of the present disclosure to provide a wire structural support joined to the proximal and distal portions of the distal flange and not the lateral portions of the distal flange, wherein the wire is configured to expand into a figure-8 or lemniscate configuration about the entire circumferential aspect of the luminal wall surface of the blood vessel and support the distal flange against the luminal wall surface of the blood vessel.

It is still another object of the present disclosure to provide an axial compression ring or cuff that engages about a circumference of the access graft, is translatable along a longitudinal axis of the access graft and into abutting relationship with an abluminal wall of the blood vessel and exerts an axially compressive force between the axial compression ring or cuff, the distal flange, and the wall of the blood vessel to impart hemostasis at the access site of the blood vessel.

It is yet another further object of the present disclosure to provide an access device and assembly that is configured for peripheral arterial access, such as through the axillary, innominate, iliac, or common femoral arteries.

It is yet another object of the present disclosure to provide axillary artery access for introducing, retaining, and withdrawing an AF-VAD into and from the axillary artery.

These and other objects, features, and advantages of the present disclosure will be more apparent to one of skill in the art from the following more detailed description of the access device and assembly taken with reference to the accompanying figures.

### BRIEF DESCRIPTION OF THE DRAWINGS

FIG. 1 is a diagrammatic illustration of an access graft joined in an angled end-to-side anastomosis to a peripheral blood vessel in accordance with the access device and assembly of the present disclosure.

FIG. 2 is a diagrammatic illustration showing an angled end-to-side anastomosis of an access graft, distal flange, and reinforcement member of the access device and assembly according to the present disclosure.

FIG. 3 is a diagrammatic view of a variant of an angled end-to-side anastomosis of an access graft, distal flange, reinforcement member, and axial compression ring or cuff of the access device and assembly according to the present disclosure.

FIG. 4 is a diagrammatic view of another variant of an angled end-to-side anastomosis of an access graft, distal flange, reinforcing member, and axial compression ring or cuff of the access device and assembly according to the present disclosure.

FIG. 5 is a diagrammatic view of still another variant of an angled end-to-side anastomosis of an access graft, distal flange, and reinforcing member of the access device and assembly according to the present disclosure.

FIG. 6 is a diagrammatic view of another variant of an access graft, distal flange and reinforcement member having two arcuate hemi-ovular reinforcement wires associated with the distal flange in accordance with the access device and assembly of the present disclosure.

FIG. 7 is a diagrammatic view of a variant of the reinforcing member in accordance with one variant of the access device and assembly of the present disclosure.

FIG. 8 is a diagrammatic view of an angled end-to-side anastomosis depicting a variant of the access graft and an asymmetric reinforced distal flange and reinforcing member in accordance with the access device and assembly of the present disclosure.

FIG. 9a is a diagrammatic illustration of another variant of the access device and assembly according to the present disclosure illustrating a two-piece variant of the access device.

FIG. 9b is a diagrammatic illustration of the two-piece variant of the access device in its assembled state.

FIG. 9c is a diagrammatic illustration of the two-piece variant of the access device in its assembled state and joined with a blood vessel in an end-to-side anastomosis.

FIG. 10 is a diagrammatic illustration of another variant of the access device in an exploded state illustrating an embodiment of an axial compression cuff.

FIG. 11 is a distal end elevational view of the access graft and distal flange in accordance with the present disclosure.

FIG. 12 is a diagrammatic view of a variant of an access graft having a side-arm branch in accordance with the access device and assembly of the present disclosure.

FIGS. 13a to 13c are sequential diagrammatic views of an everting end-side anastomosis access and closure ring in accordance with a variant of the access device and assembly of the present disclosure.

FIGS. 14a to 14d are sequential diagrammatic views of the everting end-side anastomosis access and closure ring coupled to vascular tissue and showing the sequential steps of vascular access site eversion and coupling to the access and closure ring.

### DETAILED DESCRIPTION OF THE PREFERRED EMBODIMENTS

For purposes of clarity, the following terms used in this patent application will have the following meanings:

The terminology used herein is for the purpose of describing example embodiments only and is not intended to be limiting. As used herein, the singular forms “a,” “an.” and “the” may be intended to include the plural forms as well, unless the context clearly indicates otherwise. The terms “comprises,” “comprising,” “including.” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the order discussed or illustrated, unless specifically identified as an order of performance. It is also to be understood that additional or alternative steps may be employed.

When an element or layer is referred to as being “on,” “engaged,” “connected.” or

“coupled” to or with another element, it may be directly on, engaged, connected, or coupled to the other element or layer, or intervening elements or layers may be present. In contrast, when an element is referred to as being “directly on.” “directly engaged to,” “directly connected to,” or “directly coupled to” or with another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than, more than or equal to total. For example, “substantially vertical” may be less than, greater than, or equal to completely vertical.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

Recitation of ranges of values herein are merely intended to serve as a shorthand method of referring individually to each separate value falling within the recited range, unless otherwise indicated herein, and each separate value is incorporated into the specification as if it were individually recited herein.

References to “embodiment” or “variant”, e.g., “one embodiment,” “an embodiment,” “example embodiment,” “various embodiments,” etc., may indicate that the embodiment(s) or variant(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment or variant, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The terms “configured to” or “adapted to” are used synonymously and are intended to mean that a recited structure is intended to perform a particular recited function or assume a particular recited configuration and is not merely capable thereof.

The terms “proximal” or “distal” are intended to be relative positional references and are used with reference either to a direction of blood flow relative to a device or device component or with reference to a longitudinal axis of a device or device component. For example, with reference to the graft component, the proximal end of the graft component furthest away from the major vessel or anatomic passageway, whereas the distal end of the graft is the end closest to the major vessel or anatomic passageway.

The term “saddle-shape” when used in connection with an element is intended to mean a generally hyperbolic paraboloid structure.

The term “graft” is intended to refer to any type of polymeric, biological, composite or metal tubular structure.

The term “anatomic passageway” is intended to refer to any anatomical structure having a lumen. Examples of anatomic passageways are blood vessels, the gastrointestinal track, including the esophagus, stomach, small intestine, large intestine, and rectum, or airway passages, such as the trachea and bronchi.

The terms “peripheral blood vessel” is used herein to reference the blood vessels outside the heart. It is intended that the term includes arteries, such as, for example, as the axillary, innominate, iliac, and/or femoral arteries, as well as their corresponding veins.

The term “flange” is intended to refer to any type of radially extending projection, including, without limitation, a projection that extends less than or equal to 360 degrees relative to the element that the projection extends from. Further, a flange may have a longitudinal component to its projection orientation relative to the element that the projection extends from.

This detailed description of exemplary embodiments references the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not for purposes of limitation.

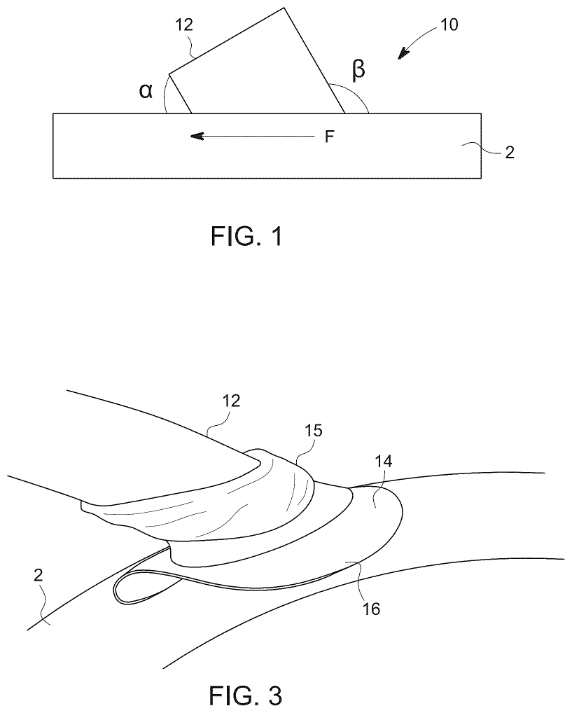

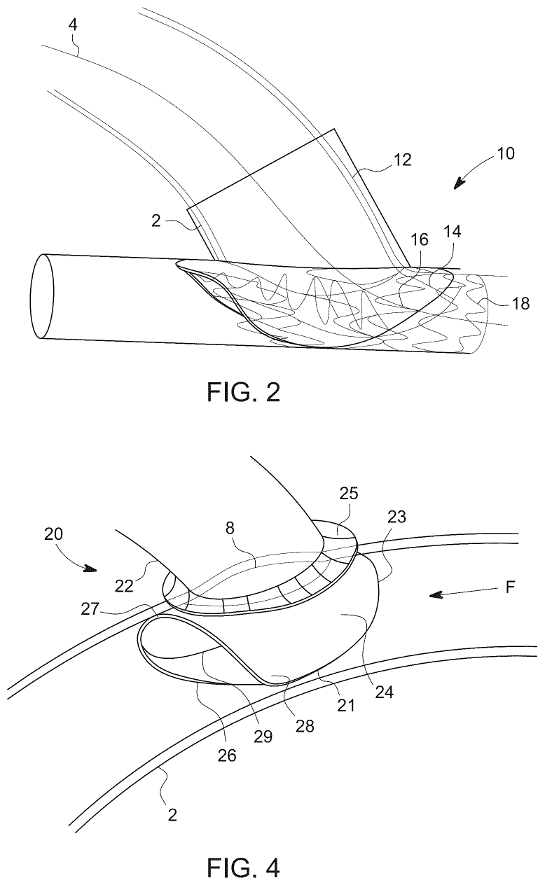

Variants and sub-variants of the access devices and assemblies of the present disclosure are illustrated in the accompanying figures. FIGS. 1 and 2 diagrammatically illustrates the fundamental configuration of the access device and assembly 10 (hereinafter “access device 10”) of the present disclosure. Access device 10 is configured to couple to a blood vessel 2, particularly a peripheral blood vessel, such as an axillary artery. Access device 10 consists generally of an access graft 12 having at least one distal flange 14 that projects radially outward from a central longitudinal axis of the access graft 12. Further, a reinforcing member 16 is provided in association with the at least one distal flange 14 and is adapted to diametrically expand the at least one distal flange 14 after the at least one distal flange 14 is positioned within a lumen of the blood vessel 2 and allow the at least one flange 14 to nest against and abut a luminal wall surface of the blood vessel 2.

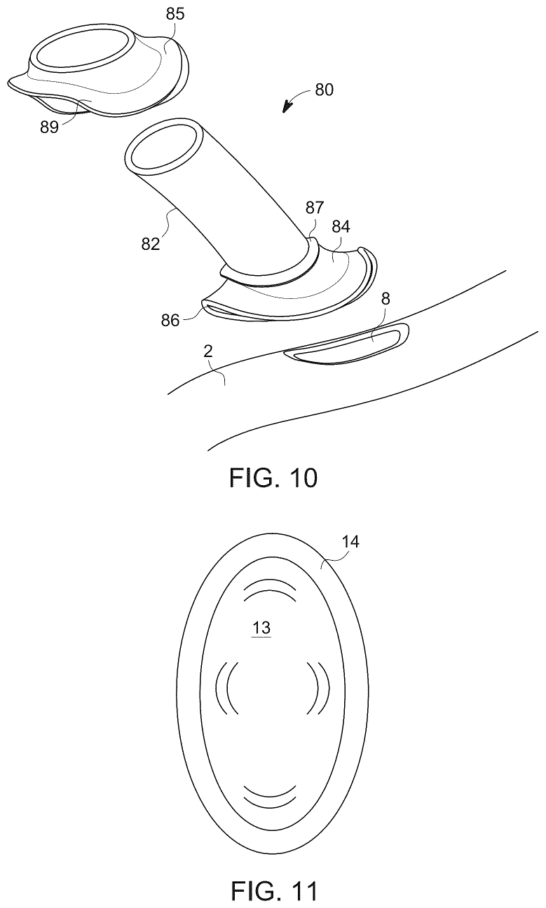

Access graft 12 is configured such that the at least one distal flange 14 is positioned at a distal end of the access graft 12 and is angularly displaced from a longitudinal axis of the access graft 12. The angular displacement of the at least one distal flange 14 forms a non-circular distal end opening 13 of the access graft 12 where it joins the blood vessel 2, as illustrated in FIG. 10. This non-circular distal end opening 13 of the access graft 12 may be elliptical or ovular in transverse cross-sectional shape and has a major axis and a minor axis, with the major axis of the non-circular distal end opening 13 adapted to be positioned parallel to a longitudinal axis of the blood vessel 2 and the minor axis positioned about a circumferential axis of the blood vessel 2. In this manner, the non-circular distal end opening 13 defines and open surface area well-suited for introduction of a medical device, such as a catheter-based AF-VAD, into and through the non-circular distal end opening 13 and facilitates traversing the bend from the access graft 12 to the lumen of the blood vessel 2.

As shown in FIGS. 1 and 2, the angular displacement of the at least one flange 14 from the longitudinal axis of the access graft 12 imparts a non-perpendicular angular orientation of the access graft 12 relative to the longitudinal axis of the blood vessel 2. This non-perpendicular angular orientation of the access graft 12, forms an acute angle α at the distal or heel region of the access graft 12 and an obtuse angle β at the proximal or toe region of the access graft 12. In this case the distal or heel region and the proximal or toe region are determined with reference to the axis of blood flow F through the blood vessel 2. Angle α is preferably between about 30-degrees to about 40-degrees to achieve a desirable approach angle for delivery of a catheter-based medical device into the blood vessel 2.

Access graft 12 and the at least one distal flange 14 may be made of synthetic polymers, such as expanded polytetrafluoroethylene (ePTFE), polyethylene terephthalate (PET), polyester, polyurethane, polylactic acid, polyglycolic acid, or nylon, xenografts, allografts, or autografts. The at least one distal flange 14 is preferably unitary with the access graft 12 and not a separate, attached component from the access graft 12; this configuration avoids a potential failure point of the access graft 12.

Reinforcing member 16 may have a wide variety of configurations. As shown in FIG. 2, the reinforcing member 16 is associated with the at least one distal flange 14 by embedding, encapsulating, joining, or otherwise attaching the reinforcing member 16 to the at least one distal flange 14. The reinforcing member 16 may be coupled to a peripheral surface or edge of the at least one distal flange 14, may be coupled to a radial surface or surfaces of the at least one distal flange 14, or may be embedded or encapsulated in the material of the at least one distal flange 14. The reinforcement member 16 may be made of a pliable or elastic polymeric material that has a greater durometer and/or a lower modulus of elasticity than the material of the distal flange, may be an elastic member, such as stainless-steel wire, or may be a shape memory polymer or metal capable of being shape set to a desired configuration, such as nickel-titanium alloys.

The reinforcement member 16 may be a latticed structure, a wire, plural wires, filaments, or other associated with the at least one distal flange 14. The reinforcement member 16 may be coupled to the at least one distal flange 14 in discrete portions of the at least one distal flange 14, about an entire periphery of the at least one distal flange 14, or about an entire circumferential portion of the at least one distal flange 14. It is important that the reinforcement member 16 be coupled to the at least one distal flange 14 in such a manner as it facilitates diametric expansion of the at least one distal flange 14 in a manner that permits the at least one distal flange 14 to seat against the luminal wall surface of the blood vessel 2, positionally secures the access graft 12, and allows for the blood vessel 2 to remain patent.

An optional luminal structural support 18 may also be provided within the lumen of the blood vessel 2 to aid in supporting the patency of the blood vessel 2 and assist in creating a passageway within the blood vessel 2 for the medical device to be passed within the blood vessel 2. The luminal structural support 18 may be, for example, a tubular or bifurcated stent device.

A variant of the access graft 10 is depicted at FIG. 3 in which there is provided an optional axial compression ring or cuff 15 that is concentrically positioned about access graft 12 and is placed in abutting relationship with the abluminal wall surface of the blood vessel 2. After the at least one flange 14 and the reinforcing member 16 are deployed within the lumen of the blood vessel, the axial compression ring or cuff 15 is translated along the access cuff and positioned to bear against the abluminal wall surface of the blood vessel. In this manner there is a distally oriented force applied by the axial compression ring of cuff 15 against the abluminal wall surface of the blood vessel 2 and a proximally oriented forced applied by the at least one flange 14 and the reinforcing member 16 against the luminal wall surface of the blood vessel 2. It will be appreciated, that this configuration positions and compresses the wall of the blood vessel 2 between the axial compression ring or cuff 15 and the at least one distal flange 14. In this manner, a hemostatic seal is created at the access site through the blood vessel 2.

As depicted in FIG. 3, the reinforcing member 16 is a circumferential wire coupled to a peripheral surface of the at least one distal flange 14. The circumferential wire reinforcing member 16 is configured to diametrically expand and urge the at least one distal flange 14 into a configuration that abuts the luminal wall surface of the blood vessel 2.

A variant of the access device and assembly 20 is illustrated in FIG. 4. Like access device and assembly 10, this variant of the access device and assembly 20 is characterized by having an access graft 22, at least one distal flange 24, and a reinforcing member 26. Additionally, there is provided an axial compression ring or cuff 25 that is circumferentially positioned about an outer circumference of the access graft 22 and is translatable along a longitudinal axis of the access graft 22 to exert an axially compressive force to the abluminal wall of the blood vessel and to the at least one distal flange 24. Unlike access device and assembly 10, the reinforcing member 26 of access device and assembly 20 consists of a wire that is configured into a FIG. 8 or lemniscate configuration and coupled only to a proximal portion 23 and a distal portion 27 of the at least one distal flange 24, leaving the lateral portions of the at least one flange not coupled to the reinforcing member 26. The proximal portion 23 and distal portion 27 of the at least one flange are oriented parallel to the longitudinal axis of the blood vessel. The uncoupled portion of the reinforcing member forms an overlapping portion 28 of the reinforcing member 26 and circumscribes a circumferential aspect of the luminal wall of the blood vessel, forming a proximal loop 21 and a distal loop 29 that are open to blood flow F there through. This FIG. 8 or lemniscate configuration of the reinforcing member 26 also helps to abut the at least one distal flange 24 against the luminal wall surface of the blood vessel 2 at the access site 8.

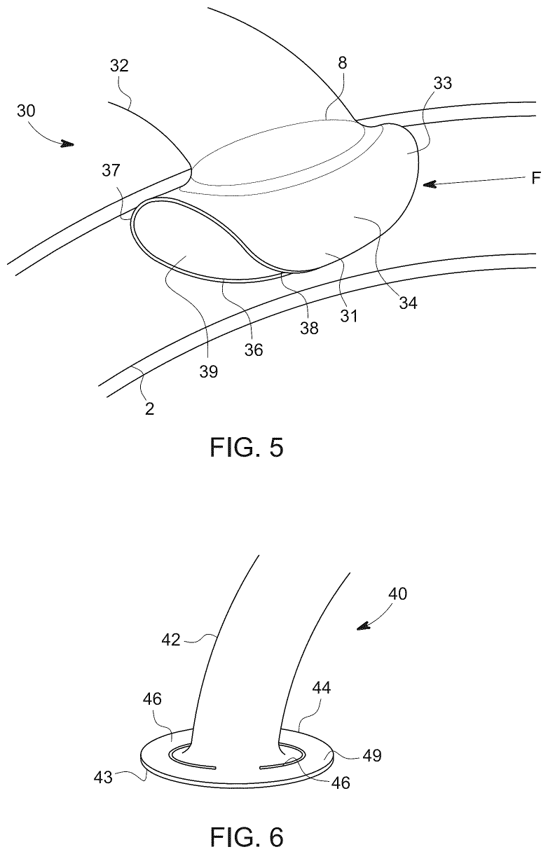

A variant of the access device and assembly 20 is access device 30 illustrated in FIG. 5. Access device 30 is identical to access device 20, in that it has an access graft 32, at least one distal flange 34 at a distal end of the access graft, where the at least one distal flange 34 is angularly displaced from the longitudinal axis of the access graft 32. When placed within the lumen of the blood vessel 2, the reinforcing member 36 is a looped wire that expands to deploy the at least one distal flange 34 within the blood vessel 2 and is coupled to the at least one distal flange 34 at proximal and distal portions of the reinforcing member only. The reinforcing member 36, when deployed within the access site 8 and the lumen of the blood vessel 2, assumes a FIG. 8 or lemniscate configuration in which there is an overlap 38, a proximal loop 31 and a distal loop 39, again relative to the direction of blood flow F through the blood vessel. The difference between access device 20 and access device 30 is that in access device 30, no axial compression ring or cuff is employed and the expansive force of the reinforcing member 36 is sufficient to maintain hemostasis with the at least one flange being pressed against the luminal wall surface of the blood vessel at the access site 8.

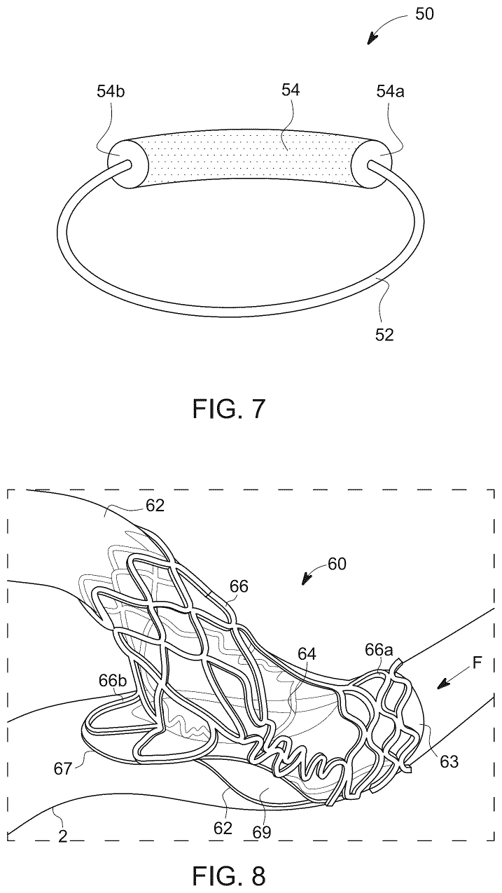

Turning now to FIGS. 6 and 7, there is illustrated access device 40 and axial compression ring or cuff 50. Access device 40 consists of an access graft 42 having at least one distal flange 44, and at least one reinforcing member 46. Like the prior described variants of the inventive access device and assembly, the access graft 42 is angularly displaced from the at least one distal flange 44 to form a non-circular distal end opening of the access graft 42 at the at least one distal flange 44. In this variant of the access device 40, the reinforcing member 46 consists of two or more arcuate reinforcing members 46 coupled to the at least one distal flange 44 and positioned at proximal 43 and distal 49 portions, respectively, of the at least one distal flange 44. The two or more arcuate reinforcing members 46 are coupled in such a manner as to reinforce the proximal 43 and distal 49 portions of the at least one distal flange 44 such that the at least one distal flange 44 is configured to abut against the luminal wall of the blood vessel 2 around the access site 8.

The axial compression ring or cuff 50 consists of an elongate wire member 52 having opposing ends (not shown) and a tubular member 54 having open ends 54a and 54b that each receive one of the opposing ends of the elongate wire member 52 therein. In this manner, the elongate wire member 52 assumes a generally circular or ovular shape while the elongate wire member 52 is constrained by the tubular member 54. The opposing ends of the elongate wire member 52 are free to move within the tubular member 54 to allow for diametric expansion and contraction of the elongate wire member The elongate wire member 42 is, therefore, and/or the tubular member 54.

In use, once the at least one distal flange 44 is deployed through the access site 8, the axial compression ring or cuff 54 is concentrically positioned about the outer circumference of the access graft 52 and translated into proximity of the abluminal wall of the blood vessel. In this position, the axial compression ring or cuff 54 exerts an axially compressive force to the at least one distal flange 44 against the luminal wall of the blood vessel 2.

FIG. 8 illustrates another variant of the access device 60. According to this variant, access device 60 is characterized by having a distal flange 64 having a generally T-shape in which there is proximal projecting flange 63 and a distal projecting flange 67. The proximal projecting flange 63 and the distal projecting flange 67 are asymmetric relative to one another. Reinforcing member 66 supports the distal flange 64, including the proximal projecting flange 63 and the distal projecting flange 67. The proximal projecting flange 63 forms a toe portion of the access device 60 and extends about at least a substantial circumferential extent of the blood vessel 2 lumen and forms a blood flow pathway 69 through the proximal projecting flange 63. A proximal section 66a portion of the reinforcing member 66 supports the proximal projecting flange 63 and applies an expansive force to abut the distal flange 64 against the luminal wall surface of the blood vessel 2. The distal projecting flange 67 forms a heel portion of the access device 60 and is supported by a distal section 66b of the reinforcing member 66. The distal section 66b applies an expansive force to abut the distal projecting flange 67 against the luminal wall surface of the blood vessel and proximate to the access site 8.

The proximal projecting flange 63 is configured to extend proximally, or upstream relative to blood flow F, within the blood vessel 2, extends about at least a substantial circumferential extent of the lumen of the blood vessel 2, and forms a blood flow pathway 69 through the proximal projecting flange 63. The distal projecting flange 67 extends about less than a 180-degree arc about the circumference of the lumen of the blood vessel and project distally relative to the blood flow F and the access site 8.

The reinforcing member 66 may, optionally, have a proximal section that is concentric with a portion of the access graft 62 that projects outward from the access site in the blood vessel 2. In this configuration, the reinforcing member 66 also supports the angular displacement of the access graft 62 relative to the distal flange 64. In this manner, introduction of a catheter-based medical device into and through the access graft 62 is oriented to guide the medical device toward and into the proximal projecting flange 63 to guide the medical device into the proximal portion of the blood vessel 2.

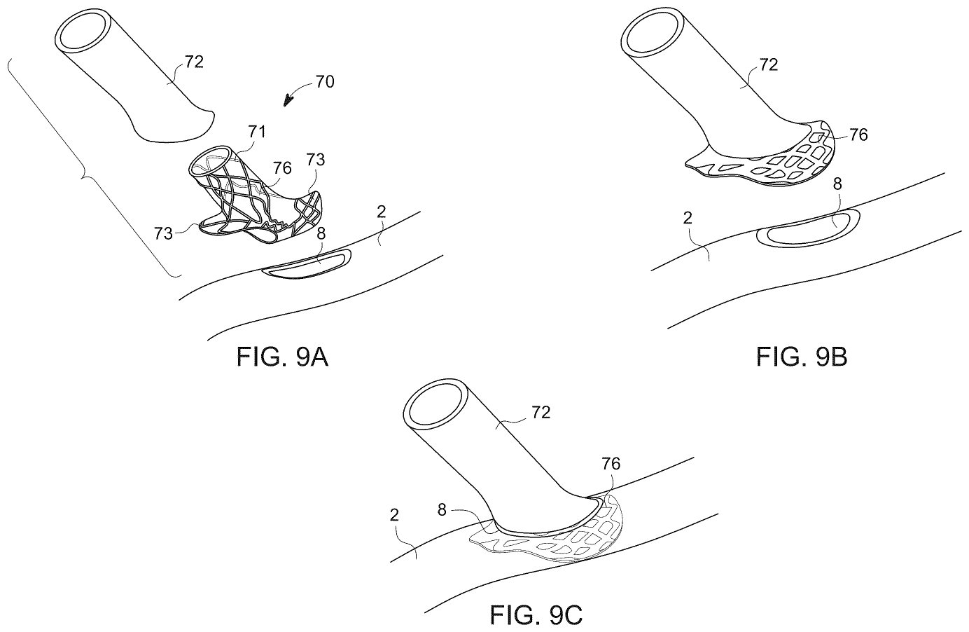

A two-piece variant 70 of the access device and assembly of the present disclosure is illustrated in FIGS. 9a-9c. The two-piece variant 70 includes a covered stent-like reinforcing member 76 that is similar to reinforcing member 66 in that it is a lattice structure having a covering that is configured into a generally T-shaped configuration having a proximal projecting flange 73, a distal projecting flange 77, and a tubular projection 71 that is configured to project out from the access site 8 in the blood vessel when coupled in and end-to-side anastomosis with the blood vessel 2, as depicted in FIG. 9c. The proximal projecting flange 73 forms a toe portion of the access device 70 and the distal projecting flange 77 forms a heel portion of the access device 70, with both the toe portion and the heel portion being disposed within the access site 8 when the end-to-side anastomosis is formed. The tubular projection 71, that joins with access graft 72, projects laterally outward from the access site 8 in the blood vessel 2.

The proximal projecting flange 78 preferably extends about at least a substantial circumferential extent of the blood vessel 2 lumen and forms a blood flow pathway through the proximal projecting flange 73. In order to circumferentially support the lumen of the blood vessel 2, it may be desirable to form a portion of the proximal projecting flange 73 as a tubular section that diametrically expands to abut the entire circumference of the blood vessel 2 lumen.

In use, the access graft 72 may be joined to the tubular projection 71 prior to forming the end-to-side anastomosis with the access device and assembly 70. Alternatively, the reinforcing member may be delivered into and through the access site 8 and expanded to seat against the blood vessel 2 lumen with the tubular projection 71 projecting laterally outward from the access site 8, then the access graft 72 is delivered and coupled to the tubular projection 71. This two-piece configuration of the access device and assembly 70 allows for use of conventional and different access grafts 72 to be used and coupled to the reinforcement member 76.

A further variant 80 of the access device and assembly of the present disclosure is shown in FIG. 10. According to variant 80, access graft 82 has at least one distal flange 84 that is reinforced by a reinforcing member 86, an engagement member 87 proximal to the at least one distal flange 84 on the access graft 82, and an axial compression cuff 85. Engagement member 87 may be any of a wide variety of engagements configured to cooperate with a mating engagement on the axial compression cuff 85. Engagement member 87 may be circumferential or concentric about the access cuff 82, and may be a projection, detent, recess, threads, interference fits, or the like. In accordance with a contemplated embodiment, the engagement member is a fluorinated ethylene propylene (FEP) ring or helix on an outer circumferential surface near the distal end of the access graft 82. The axial compression cuff 85 has an annular opening that couples concentrically with the outer circumferential aspect of the access cuff and is configured to engagement with engagement member 86. Axial compression cuff 85 may, optionally, have one or more distally extending flanges 89 that are configured to nest against an abluminal wall of the blood vessel and, via interface with engagement member 87, exert an axially compressive force against the abluminal surface of the blood vessel 2 about the access site 8, at least one flange 84, and the reinforcing member 86, with the blood vessel 2 being between the axial compression cuff 85 and the at least one flange 84. This configuration and the axial compression creates a hemostatic seal about the access site 8 without the need for suturing.

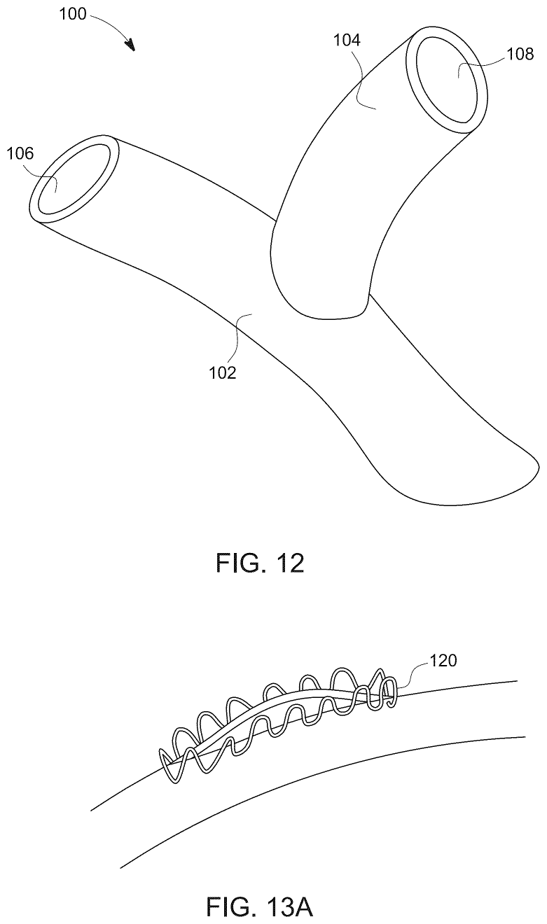

Finally, as shown in FIG. 12, a variant 100 of the access graft 102 is shown in which the access graft 102 having a primary lumen 106 has one or more side-arm conduits 104 having a secondary lumen 108 that is in fluid flow communication with the primary lumen 106. Variant 100 permits a single access point in the blood vessel to accommodate both placement of a medical device, such as an AF-VAD, through the primary lumen 108 and simultaneously allow for connection of cardiopulmonary bypass or an ECMO cannular for arterial inflow through the secondary lumen 108.

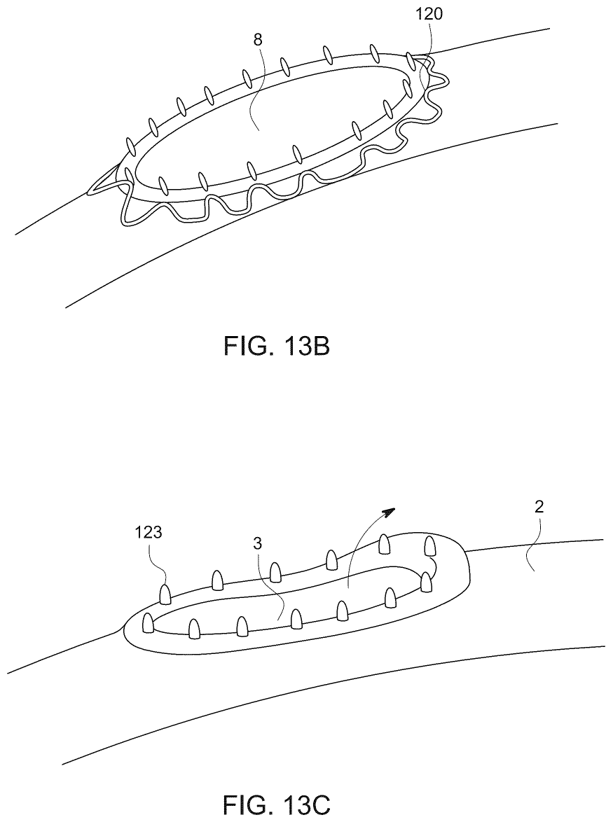

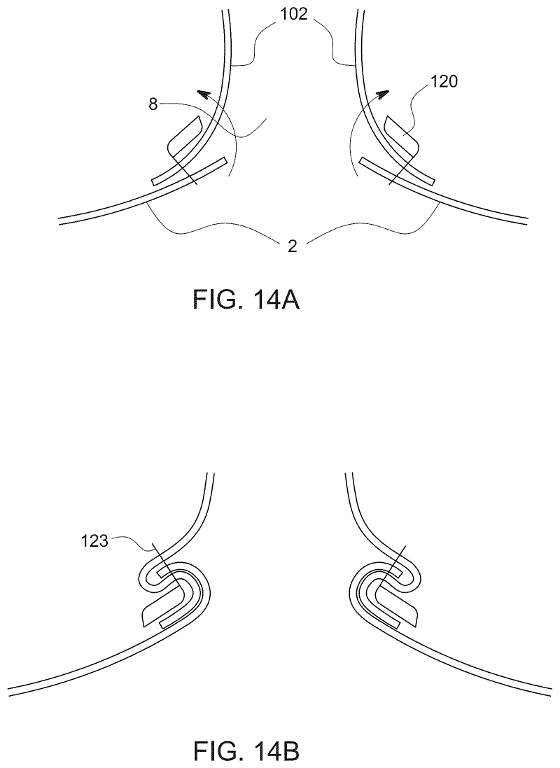

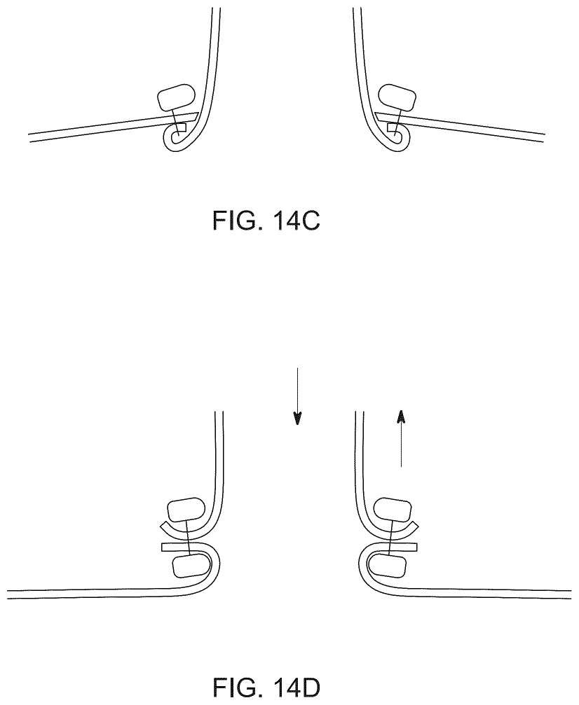

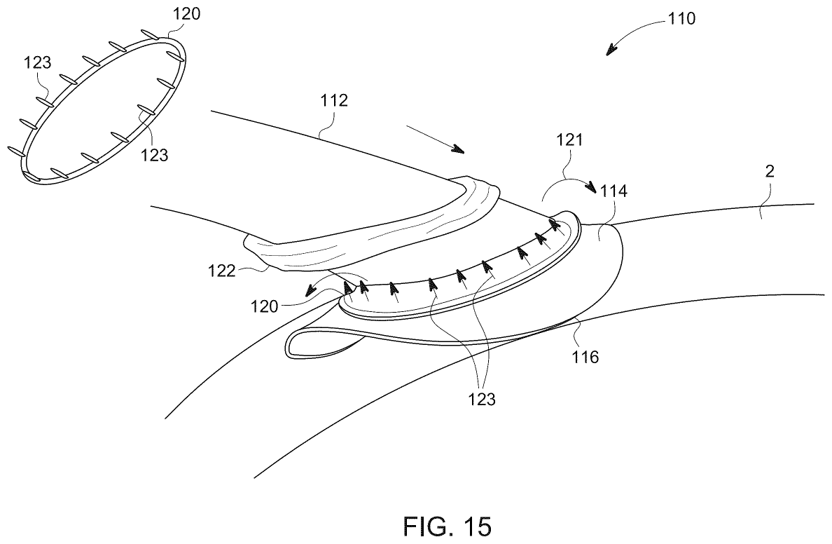

A further access device and assembly 110 is illustrated in FIGS. 13a to 14d. Access device and assembly 110 is graft agnostic in that it is configured for use with or without an access graft. Access device and assembly 110 consists generally of a ring 120 having a plurality of plurality of tines 123 arrayed about a circumference of ring 120 and initially projecting parallel to a longitudinal axis of the ring 120. Ring 120 is preferably made of a shape memory material, such as a shape memory metal alloy or a shape memory polymer. The programmed shape of ring 120 allows for the ring 120 to be diametrically expandable and capable of annularly everting about its longitudinal axis or of a fixed diameter and capable of annularly everting about its longitudinal axis. By configuring the ring 120 to be diametrically expandable, the ring 120 is adapted to expand to allow a catheter-based medical device to be passed through ring 120, access site 8, and into the lumen of blood vessel 2. Moreover, diametrically expandable ring 120 will then diametrically contract about an outer circumference of the catheter-based medical device to impart a seal about the catheter-based medical device. The everting characteristic of ring 120 allows the plurality of tines to have a first state in which they are oriented distally and configured to engage with the abluminal wall tissue of the blood vessel, with the annulus of ring 120 open to allow for the blood vessel 2 tissue to be pulled through the ring 120 with the plurality of tines 123 rotating about their axis as the blood vessel 2 tissue is pulled through the annulus. Then, upon opening the vascular access site, such as by a venotomy or arteriotomy, the plurality of tines 123 will complete their eversion and be oriented in their second state where the plurality of tines 123 have rotated about 180 degrees and oriented proximally with the vascular tissue embedded by the plurality of tines 123 now being everted such that abluminal wall of the blood vessel is everted upon itself and the luminal wall surface of the blood vessel 2 is exposed with the plurality of tines 123 protruding from the exposed luminal wall surface.

The ring 120 may be used with or without a graft. FIGS. 13a-13c illustrate the sequential steps of engaging the plurality of tines 123 of ring 120 with the blood vessel with the plurality of tines distally oriented to engage the abluminal wall of the blood vessel, then pulling the abluminal surface of the blood vessel to become the vascular access site 8 through the ring 120, and creating the vascular access site 8 by arteriotomy or venotomy, and allowing the ring 120 to evert such that the plurality of tines 123 become oriented in an opposite and proximal orientation. FIGS. 14a-14d illustrate the sequential steps of joining a graft 102 to a blood vessel 2 about an access site 8, with the eversion of the ring 120 such that the plurality of tines 123 rotate from a distal orientation toward the lumen of the blood vessel 2 to a proximal orientation away from the lumen of the blood vessel and parallel to the longitudinal axis of the graft 102.

FIG. 15 further depicts an assembly 120 in which the ring 120 is engaged about a distal circumference of a graft 112 and proximal to a distal flange 114 at the distal end of the graft 112. When everted 121, the plurality of tines form the eversion in the blood vessel 2 about the circumference of ring 120 and about the circumference of the access site. The distal flange 114 is supported by reinforcement member 116 against the luminal wall surface of the blood vessel 2. An axial compression member 122 is concentrically engaged about the graft 112 and brought into engagement with the plurality of tines 123 to both cover the plurality of tines 123 and exert an axially compressive force to the ring 120 and the reinforcement member 116 coupled to the distal flange 114, which seals the access site between the axial compression member 122 and the distal flange 114 and allows communication between the graft 112 and the lumen of the blood vessel 2.

In a variant of assembly 120, an engagement member (not shown) similar to engagement member 87 in FIG. 10 may be provided proximate to the distal end of the graft 112. The engagement member may be circumferential or concentric about the access graft 112, and may be a projection, detent, recess, threads, interference fits, or the like. In accordance with a contemplated embodiment, the engagement member is a fluorinated ethylene propylene (FEP) ring or helix on an outer circumferential surface near the distal end of the access graft 112. The axial compression member 122 has an annular opening that couples concentrically with the outer circumferential aspect of the access graft 112 and is configured to engage with the engagement member. Axial compression member 122 may, optionally, have one or more distally extending flanges that are configured to nest against an abluminal wall of the blood vessel and, via interface with the engagement member, exert an axially compressive force against the abluminal surface of the blood vessel 2 about the access site 8. This configuration and the axial compression creates a hemostatic seal about the access site 8 without the need for suturing.

While the present disclosure is made with reference to certain variants and examples of the access graft and assembly, it will be understood and appreciated by those skilled in the art that variations in materials, configuration, construction, dimensions, or uses are contemplated and intended within the scope of the present disclosure, which is intended to be limited only by the claims appended hereto.

1. A sutureless end-to-side anastomosis system, comprising:
a. An access graft having a distal end having a non-circular transverse cross-sectional profile;
b. A flange at the distal end of the access graft; and
c. A reinforcing member associated with the flange at the distal end of the graft.

2. The sutureless end-to-side anastomosis system of claim 1, wherein the reinforcing member and the flange are configured to pass into a lumen of a blood vessel and abut against a luminal wall surface of the blood vessel.

3. The sutureless end-to-side anastomosis system of claim 2, wherein the blood vessel is a peripheral artery.

4. The sutureless end-to-side anastomosis system of claim 1, further comprising a tubular graft configured to operably engage with the access graft through a vascular wall without suture engagement between the tubular graft and the access graft.

5. The sutureless end-to-side anastomosis system of claim 4, wherein the tubular graft further comprises an expandable stent.

6. The sutureless end-to-side anastomosis system of claim 1, wherein the distal end of the access graft and the flange have an acute angle relative to a longitudinal axis of the access graft.

7. The sutureless end-to-side anastomosis system of claim 6, wherein the acute angle is between about 30 degrees to about 40 degrees relative to the longitudinal axis of the access graft.

8. The sutureless end-to-side anastomosis system of claim 1, wherein the reinforcing member further comprises at least one semi-ovular wire ring member.

9. The sutureless end-to-side anastomosis system of claim 1, wherein the reinforcing member further comprises a shape memory wire and a shape memory tube, wherein the shape memory wire has opposing ends that are engaged within the shape memory tube and movable therein.

10. The sutureless end-to-side anastomosis system of claim 1, wherein the reinforcing member further comprises at least one wire configured into one or more wire loops capable of diametrically expanding the flange of the access graft.

11. The sutureless end-to-side anastomosis system of claim 1, wherein the flange further comprises an asymmetric flange having a heel portion and a toe portion, wherein the heel portion and the toe portion project in opposite directions from one another.

12. The sutureless end-to-side anastomosis system of claim 11, wherein the heel portion further comprises a section that subtends less than a 180-degree arc relative to a blood vessel lumen.

13. The sutureless end-to-side anastomosis system of claim 11, wherein the toe portion comprises a section that subtends greater than a 180-degree arc relative to a blood vessel lumen.

14. The sutureless end-to-side anastomosis system of claim 1, further comprising a circumferential constraining member operably connected with the access graft, the circumferential constraining member being configured to allow a medical device to pass into and through the access graft and circumferentially seal the access graft against the medical device.

15. The sutureless end-to-side anastomosis system of claim 14, wherein the medical device is a circulatory assist device.

16. The sutureless end-to-side anastomosis system of claim 15, wherein the circulatory assist device is an axial flow ventricular assist device.

17. A sutureless end-to-side anastomosis assembly, comprising:
a. A reinforcing member comprising a covered stent graft having a tubular projection, a proximal projecting flange, and a distal projecting flange latticed associated with the flange at the distal end of the graft, wherein the reinforcing member is configured to be placed through an access opening in a blood vessel and expand within the blood vessel and seat the reinforcing member within the lumen of the blood vessel; and
b. An access graft having a distal end having a non-circular transverse cross-sectional profile, the access graft being removably coupled to the tubular projection of the reinforcing member.

18. The sutureless end-to-side anastomosis assembly of claim 17, wherein the blood vessel is a peripheral artery.

19. The sutureless end-to-side anastomosis assembly of claim 18, wherein the tubular projection of the reinforcing member is positioned at a non-orthogonal angle relative to the distal projecting flange.

20. The sutureless end-to-side anastomosis assembly of claim 19, wherein the non-orthogonal angle is between about 30 degrees to about 40 degrees.

21. The sutureless end-to-side anastomosis assembly of claim 17, wherein the proximal projecting flange and the distal projecting flange are asymmetric relative to one another.

22. The sutureless end-to-side anastomosis assembly of claim 21, wherein the heel portion further comprises a section that subtends less than a 180-degree arc relative to a blood vessel lumen.

23. The sutureless end-to-side anastomosis assembly of claim 21, wherein the toe portion comprises a tubular section that subtends greater than a 180-degree arc relative to a blood vessel lumen.

24. The sutureless end-to-side anastomosis assembly of claim 23, wherein the tubular portion subtends an entire circumferential extent of the blood vessel lumen.

25. The sutureless end-to-side anastomosis assembly of claim 17, wherein the access graft further comprises a side-arm branch in fluid flow communication with a primary lumen in the access graft.
